(19) United States
(12) Patent Application Publication
(10) Pub. No.: US 2004/0127479 A1
(43) Pub. Date: Jul. 1, 2004

(54) Peroxynitrite rearrangement catalysts

(75) Inventors: Gisbert Depke, Berlin (DE); Margrit Hillmann, Berlin (DE); Guenter Michl, Ruedersdorf (DE); Johannes Platzek, Berlin (DE); Detlev Suelzle, Berlin (DE); Roland Neuhaus, Berlin (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: Merck Patent GmbH, Darmstadt (DE)

(21) Appl. No.: 10/648,800

(22) Filed: Aug. 27, 2003

Related U.S. Application Data
(60) Provisional application No. 60/461,417, filed on Apr. 10, 2003.
(60) Provisional application No. 60/406,985, filed on Aug. 30, 2002.

(30) Foreign Application Priority Data
Aug. 27, 2002 (DE) 10240343.0-44

(51) Int. Cl. A61K 31/555; C07D 487/22

(52) U.S. Cl. 514/185; 514/410; 540/145

(57) ABSTRACT

The invention relates to the use of paramagnetic 3-, 8-substituted porphyrin derivatives with various substituents in 13- and 17-positions of the porphyrin skeleton as peroxynitrite rearrangement catalysts.

This application claims the benefit of the filing date of U.S. Provisional Application Serial No. 60/406,985 filed Aug. 30, 2002 and Serial No. 60/461,417 filed on Apr. 10, 2003.

The invention relates to the use of metal-containing complexes that catalyze the rearrangement of peroxynitrite for the production of pharmaceutical agents for treating diseases.

As early as 1990, peroxynitrite was described by Beckman et al. (Beckman et al., 1990, Proc. Natl. Acad. Sci. USA. 87, 1620-1624) as a toxic metabolite, which is produced by the diffusion-controlled reaction between nitrogen monoxide (NO, nitric oxide) and superoxide anion (O₂⁻). Peroxynitrite is involved in a number of inflammatory processes that play an important role in diseases, such as, for example, Alzheimer's dementia, multiple sclerosis, and amyotrophic lateral sclerosis, and are made responsible for cellular degeneration and the induction of apoptosis.

Peroxynitrite reacts with a number of proteins by amino acid radicals being oxidized or nitrated. Nitrotyrosine radicals are increasingly found in the tissue of patients who are suffering from multiple sclerosis, since peroxynitrite ensures the nitration of tyrosine radicals of the filaments of motor neurons. A neuronal dysfunction (Estévez et al., 1999, Science 286, 2498-2500) results by the thus disrupted contraction of the filaments. A cause of the vasoconstriction that is impaired after a stroke consists in the oxidation of the lipid radicals of the cell membrane, induced by peroxynitrite, in which injuries to the endothelium and edema resulting therefrom—and the formation of neutrophils—result.

A pharmacological intervention to prevent the actions mediated by peroxynitrite can take place on the part of the starting substances (NO, and O₂⁻) or on the part of the product.

An approach on the part of the product peroxynitrite was first described by Salvemini et al. (Salvemini et al., 1998, Proc. Natl. Acad. Sci. USA., 95, 2659-2663). In this approach, peroxynitrite is rearranged by means of a catalyst into harmless end products. It is possible to convert large amounts of peroxynitrite with only small concentrations of catalyst. An advantage of this approach is based on the fact that it cannot result in the formation of disadvantageous decomposition products, such as, for example, the reactive oxygen species, and that it results in eliminating the inhibition of the superoxide-dismutase (SOD) by peroxynitrite. This treatment method with novel compounds consequently has a two-fold advantage in the treatment of diseases. Thus, on the one hand, the rate of the conversion of peroxynitrite is accelerated, and, on the other hand, the SOD is protected relative to inactivation by peroxynitrite.

As possible rearranging catalysts, metal-containing complexes are known to date (WO 95/31197, U.S. Pat. No. 6,245,758, WO 98/04132, U.S. Pat. No. 5,872,124, WO 00/75144, WO 01/26655, U.S. Pat. No. 6,372,727). The metalloporphyrins that are described by Salvemini et al. show protective action in inflammation models. (Salvemini et al., 1998, Proc. Natl. Acad. Sci. USA. 95, 2659-2663 and British J. Pharmacol., 1999, 127, 685-692). The same class of compounds was described as effective by Cuzzocrea et al. in an intestinal artery occlusion model. (Cuzzocrea et al., 2000, FASEB J. 14 (9), 1061-1072 and Cuzzocrea et al., 2001, Pharmacology Rev. 53, 135-159). Cross et al. demonstrated the effectiveness of these substances in an MS model (“experimental autoimmune encephalomyelitis”=EAE) in mice, (Cross et al., 2000, J. Neuroimmunology 107, 21-28). Mackensen et al. showed for the first time the effectiveness of a manganese-containing porphyrin in a focal ischemia model, the focal MCAO (middle cerebral artery occlusion). (Mackensen et al., 2001, J. Neurosci. 21, 4582-4592).

To date, little is known on the side effects, such as toxicity and in vivo availability, as well as the blood-cerebrospinal permeability of these known rearrangement catalysts.

For the treatment and prophylaxis of diseases that have their cause in the reactions that are mediated by peroxynitrite, the urgent problem is to provide well-tolerated, chemically stable substances (catalysts) that are available in vivo to increase the rearrangement of peroxynitrite in harmless products. These substances that are effective in vivo can be used to develop medications for treating diseases.

This invention solves the problem by providing porphyrin complexes that are used as peroxynitrite rearrangement catalysts. These porphyrins are distinguished by their good in vivo availability as well as by their chemical stability. They have already been used as agents to diagnose tumors, and their use for necrosis and infarction imaging was already disclosed in WO 00/05235.

In this invention, it was possible to show by means of NMR and UV/VIS spectroscopy that the porphyrins of WO 00/05235 according to the invention catalyze the rearrangement of peroxynitrite in harmless end products, namely nitrate and nitrite. By means of a model for cell injuries, it was possible to show that the porphyrins according to the invention are protective and protect the cells from peroxynitrite injuries, induced by the peroxynitrite donor SIN-1. These porphyrins are already very well characterized, and it is known that they have no side effects, good water solubility and a good in-vivo availability.

The use of porphyrin complexes, which have, on the one hand, a peroxynitrite-rearranging property, and, on the other hand, diagnostic properties, makes possible a specific treatment of the diseases that are caused by peroxynitrite and their diagnosis with use of imaging processes, such as, e.g., MRT.

This invention provides pharmaceutical agents for this specific treatment and relates to a porphyrin complex that consists of a ligand of general formula I

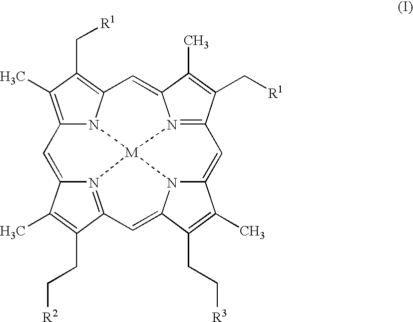

as well as at least one ion of an element of atomic numbers 20-32, 37-39, 42-51 or 57-83, in which

M stands for a paramagnetic ion,

R¹ stands for a hydrogen atom, for a straight-chain C₁-C₆-alkyl radical, a C₇-C₁₂-aralkyl radical or for a group OR′, in which R′ is a hydrogen atom or a C₁-C₃-alkyl radical,

R² stands for R³, a group —CO-Z or a group —(NH)ₒ-(A)_{q}-NH-D, in which

Z is a group —OL, with L in the meaning of an inorganic or organic cation or a C₁-C₄-alkyl radical,

A means a phenylenoxy group or a C₁-C₁₂-alkylene group or a C₇-C₁₂ aralkylene group that is interrupted by one or more oxygen atoms, o and q, independently of one another, mean the number 0 or 1, and

D means a hydrogen atom or a group —CO-A-(COOL)ₒ-(H)ₘ, with m equal to 0 or 1, and provided that the sum of m and o is equal to 1,

R³ stands for a group —(C=Q)(NR⁴)ₒ-(A)_{q}—(NR⁵)—K,

in which Q stands for an oxygen atom or for two hydrogen atoms,

R⁴ means a group -(A)_{q}—H, and

K means a complexing agent of general formula (IIa), (IIb), (IIc), (IId) or (IIe), whereby R⁵ stands for the case that K is a complexing agent of Formula (IIa) and has the same meaning as R⁴, and R⁵ stands for the case that K is a complexing agent of Formula (IIb), (IIc), (Ild) or (IIe) and has the same meaning as D,

provided that a direct oxygen-nitrogen bond is not allowed,

and K stands for a complexing agent of general formula (IIa), (IIb), (IIc), (IId), (IIe) or (IIf)

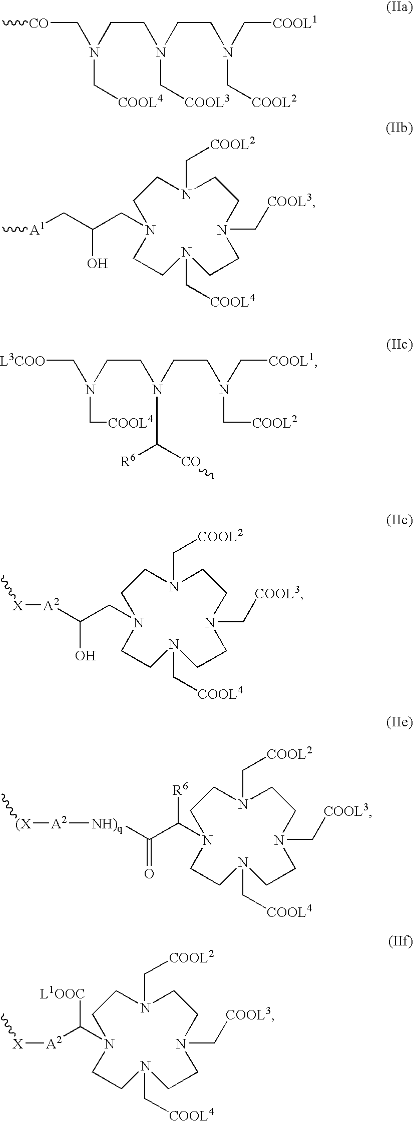

in which

q has the above-indicated meaning,

A¹ has the meaning that is indicated for A,

R⁶ stands for a hydrogen atom, a straight-chain or branched C₁-C₇-alkyl group, a phenyl or benzyl group,

A² stands for a phenylene-, —CH₂—NHCO—CH₂—CH(CH₂COOH)—C₆H₄—β-, —C₆H₄—O—(CH₂)₀₋₅-β, —C₆H₄—(OCH₂CH₂)₀₋₁—N(CH₂COOH)—CH₂-β or a C₁-C₁₂-alkylene- or C₇-C₁₂-alkylene group that is optionally substituted by one or more oxygen atoms, 1 to 3-NHCO groups or 1 to 3-CONH groups and/or substituted with 1 to 3-(CH₂)ₒ₋₅ COOH groups, whereby β stands for the binding site to X,

X stands for a —CO— or NHCS group, and

L¹, L², L³ and L⁴, independently of one another, stand for a hydrogen atom or a metal ion equivalent of an element of the above-mentioned atomic number, provided that at least two of these substituents stand for metal ion equivalents and that other anions are present to compensate for optionally present charges in the metalloporphyrin, and in which free carboxylic acid groups that are not required for complexing can also be present as salts with physiologically compatible inorganic and/or organic cations or as esters or as amides,

and that, moreover, also increase the rearrangement rate of peroxynitrite in harmless products and thus can be used for the production of a pharmaceutical agent for treatment and prophylaxis of radical-mediated cell injuries.

It was possible to show by means of NMR and UV/VIS spectroscopy that the compounds according to the invention catalyze the rearrangement of peroxynitrite in harmless end products, namely nitrate and nitrite. Peroxynitrite is a strong oxidant that is produced by the reaction of nitrogen oxide (NO) and superoxide-anion (O₂⁻). It was possible to show that NO is generated in many cells, such as, for example, in macrophages, in neutrophil cells, hepatocytes and endothelial cells. The direct reaction of NO and O₂⁻ results in the formation of the peroxynitrite ion, which quickly dissolves into oxidizing intermediate compounds under physiological conditions. These intermediate oxidation stages are responsible for the injuries to the biological targets.

The results of these damages can be associated with pathological consequences, including the oxidation and nitration of proteins, lipids and DNA. Peroxynitrite can pass through cell membranes at a significantly higher speed than other oxidants, and peroxynitrite can quickly penetrate the interior of the cell even in the presence of a biological membrane. Peroxynitrite is known for the nitration of tyrosine radicals in proteins, and it oxidizes sulfhydryl radicals, methionines and macromolecules, such as, for example, metal enzymes, DNA and lipids.

Because of its high reactivity, peroxynitrite was brought into contact with many diseases. The invention relates to the use of the compounds according to the invention for the production of a pharmaceutical agent for treatment and prophylaxis of radical-mediated cell injuries. These include neurodegenerative diseases, inflammatory diseases, autoimmune diseases, and cardiovascular diseases.

For example, there can be mentioned:

Cerebral ischemia, ischemic reperfusion disease, hypoxia and other neurodegenerative diseases that are associated with inflammations, such as multiple sclerosis, ALS (amyotrophic lateral sclerosis) and comparable sclerotic diseases, Parkinson's disease, Huntington's disease, Korksakoff's disease, epilepsy, vomiting, sleep disturbances, schizophrenia, depression, stress, pain, migraine, hypoglycemia, dementia, such as, e.g., Alzheimer's disease, HIV dementia and presenile dementia.

They are also suitable for treating diseases of the cardiovascular system, such as arteriosclerosis, and for treating autoimmune and/or inflammatory diseases, such as hypotension, ARDS (adult respiratory distress syndrome), sepsis or septic shock, rheumatoid arthritis, osteoarthritis, insulin-dependent diabetes mellitus (IDDM), inflammatory disease of the pelvis/intestine (bowel disease), meningitis, glomerulonephritis, acute and chronic liver diseases, diseases by rejection (for example allogenic heart, kidney or liver transplants) or inflammatory skin diseases such as psoriasis, etc.

The porphyrin complexes according to the invention contain as a paramagnetic ion in the porphyrin skeleton the iron(III), manganese(III), copper(II), cobalt(III), chromium(III), nickel(II) or vanadyl(II) ion, whereby the first three ions mentioned are preferred.

If one of the ions that is bonded in the porphyrin is present in a higher oxidation stage than +2, the excess charge(s) is (are) compensated for by, e.g., anions of organic or inorganic acids, preferably by acetate, chloride, sulfate, nitrate, tartrate, succinate and maleate ions or by the negative charges that are present in R² and/or R³.

The carboxyl groups that are not required for the complexing of the metal ions can optionally be present as esters, as amides, or as salts of inorganic or organic bases. Suitable ester radicals are those with 1 to 6 C atoms, preferably ethyl ester; suitable inorganic cations are, for example, the lithium ion and the potassium ion, and especially the sodium ion. Suitable cations of organic bases are those of primary, secondary or tertiary amines, such as, for example, ethanolamine, diethanolamine, morpholine, glucamine, N,N-dimethylglucamine, in particular the meglumine.

As complexing agent radical K, preferably derivatives of diethylenetriaminepentaacetic acid and 1,4,7,10-tetraazacyclododecane-1,4,7-triacetic acid can be mentioned, which are bonded via a linker to the respective porphyrin.

The production of the complex compounds of general formula I is carried out according to methods that are known in the literature (see, e.g., DE 4232925 for II a and II b; see, e.g., DE 19507822, DE 19580858 and DE 19507819 for III c; and see, e.g., U.S. Pat. No. 5,053,503, WO 96/02669, WO 96/01655, EP 0430863, EP 255471, U.S. Pat. No. 5,277,895, EP 0232751, and U.S. Pat. No. 4,885,363 for II d, II e and II f).

The production of the compound according to the invention is already described in WO 00/17205.

The compounds in which R2 and R3 stand for CONHNHK groups are preferred. The synthesis of the 3,3′-(7,12-diethyl-3,8,13,17-tetramethylporphyrin-2,18-diyl)di(propanohydrazide) that is required as an educt in this connection is described in Z. Physiol Chem. 241, 209 (1936).

The introduction of the desired metals (e.g., Mn) in the porphyrins is carried out according to methods that are known in the literature (e.g., The Porphyrins, ed. D. Dolphin, Academic Press, New York 1980, Vol. V, 459; DE 4232925), whereby essentially the following can be mentioned:

a) The substitution of the pyrrolic NHs (by heating the metal-free ligand with the corresponding metal salt, preferably the acetate, optionally with the addition of acid-buffering agents, such as, e.g., sodium acetate, in a polar solvent), or

b) The “recomplexing,” in which a metal that is already complexed by a ligand is displaced by the desired metal.

As a solvent, primarily polar solvents, such as, e.g., methanol, glacial acetic acid, dimethylformamide, chloroform and water, are suitable.

The introduction of paramagnetic metal M into the porphyrin system can be carried out before or after the linkage of complexing agent radical K. As a result, an especially flexible procedure for the synthesis of the compounds according to the invention is made possible.

The chelation of radical K is carried out in a way that is known in the literature (see, e.g., DE 34 01 052) by the metal oxide or metal salt (e.g., the nitrate, acetate, carbonate, chloride or sulfate) of the respectively desired metal being suspended or dissolved in polar solvents such as water or aqueous alcohols and being reacted with the corresponding amount of the complexing ligands. If desired, acidic hydrogen atoms or acid groups that are present can be substituted by cations of inorganic and/or organic bases or amino acids.

The neutralization is carried out in this case with the aid of inorganic bases, such as, e.g., alkali hydroxides or alkaline-earth hydroxides, -carbonates or -bicarbonates and/or organic bases such as, i.a., primary, secondary and tertiary amines, such as, e.g., ethanolamine, morpholine, glucamine, N-methylglucamine and N,N-dimethylglucamine, as well as basic amino acids, such as, e.g., lysine, arginine and ornithine or amides of originally neutral or acidic amino acids.

For the production of the neutral complex compounds, for example, enough of the desired bases can be added to, for example, the acidic complex salts in aqueous solution or suspension such that the neutral point is reached. The solution that is obtained can then be evaporated to the dry state in a vacuum. It is frequently advantageous to precipitate the neutral salts that are formed by adding water-miscible solvents, such as, for example, lower alcohols (e.g., methanol, ethanol, isopropanol), lower ketones (e.g., acetone), polar ethers (e.g., tetrahydrofuran, dioxane, 1,2-dimethoxyethane) and thus to obtain easily isolated and readily purified crystallizates. It has proven especially advantageous to add the desired base as early as during the complexing of the reaction mixture and thus to save a process step.

If the acidic complex compounds contain several free acid groups, it is often suitable to produce neutral mixed salts that contain both inorganic and organic cations as counterions.

This can happen, for example, by the complexing ligands being reacted in aqueous suspension or solution with the oxide or salt of the element that yields the central ion and half the amount of an organic base that is required for neutralization, the formed complex salt being isolated, optionally purified and then mixed with the necessary amount of inorganic base for complete neutralization. The sequence in which the base is added can also be reversed.

Another possibility for resulting in neutral complex compounds consists in converting the remaining acid groups in the complex completely or partially into esters. This can happen by subsequent reaction in the finished complex (e.g., by exhaustive reaction of the free carboxy groups with dimethyl sulfate).

The production of the pharmaceutical agents according to the invention is also carried out in a way that is known in the art by the complex compounds according to the invention—optionally with the addition of the additives that are commonly used in galenicals—being suspended or dissolved in aqueous medium and then the suspension or solution optionally being sterilized. Suitable additives are, for example, physiologically harmless buffers (such as, e.g., tromethamine), small additions of complexing agents (such as, e.g., diethylenetriaminepentaacetic acid), or, if necessary, electrolytes such as, e.g., sodium chloride or, if necessary, antioxidants, such as, e.g., ascorbic acid.

In principle, it is also possible to produce the pharmaceutical agents according to the invention without isolating the complex salts. In each case, special care must be used to perform the chelation such that the salts and salt solutions according to the invention are virtually free of uncomplexed metal ions that have a toxic effect.

This can be ensured, for example, with the aid of color indicators, such as xylenol orange, by control titrations during the production process. The invention therefore also relates to the process for the production of complex compounds and salts thereof. As a final precaution, there remains purification of the isolated complex salt.

To use the compounds according to the invention as pharmaceutical agents, the latter are brought into the form of a pharmaceutical preparation that in addition to the active ingredient for the enteral or parenteral administration contain suitable pharmaceutical, organic or inorganic inert carrier materials, such as, for example, water, gelatin, gum arabic, lactose, starch, magnesium stearate, talc, vegetable oils, polyalkylene glycols, etc. The pharmaceutical preparations can be present in solid form, for example as tablets, coated tablets, suppositories, or capsules, or in liquid form, for example as solutions, suspensions or emulsions. Moreover, they optionally contain adjuvants, such as preservatives, stabilizers, wetting agents or emulsifiers; salts for changing the osmotic pressure, or buffers.

These pharmaceutical preparations are also the subject of this invention.

For parenteral use, in particular injection solutions or suspensions, in particular aqueous solutions of the active compounds in polyhydroxyethoxylated castor oil, are suitable.

If suspensions or solutions of the agents according to the invention in water or in physiological salt solution are desired for enteral administration or other purposes, they are mixed with one or more adjuvant(s) that are commonly used in galenicals (e.g., methyl cellulose, lactose, mannitol) and/or surfactant(s) (e.g., lecithins, Tween®, Myrj®) and/or flavoring substances for taste correction (e.g., ethereal oils).

As carrier systems, surface-active adjuvants such as salts of bile acids or animal or vegetable phospholipids, but also mixtures thereof as well as liposomes or components thereof can also be used.

For oral administration, in particular tablets, coated tablets, or capsules with talc and/or hydrocarbon vehicles or binders, such as, for example, lactose, corn or potato starch, are suitable. The application can also be carried out in liquid form, such as, for example, as juice, to which optionally a sweetener is added.

The enteral, parenteral and oral administrations are also subjects of this invention.

The dosage of the active ingredients can vary depending on method of administration, age and weight of the patient, type and severity of the disease to be treated and similar factors. The daily dose is 0.5-1000 mg, preferably 50-200 mg, whereby the dose can be given as a single dose to be administered once or divided into 2 or more daily doses.

This invention also relates to the use of the porphyrin complexes according to the invention according to Formula (I) for the treatment and prophylaxis of diseases that are caused by the peroxynitrite-mediated reactions and that are weakened and/or treated by the increase in the conversion rate of peroxynitrite.

This invention relates in particular to the use of the porphyrin complexes of general formula (I) according to the invention for the treatment and prophylaxis of diseases that include neurodegenerative diseases, inflammatory diseases, autoimmune diseases, and cardiovascular diseases. For example, there can be mentioned:

Cerebral ischemia, ischemic reperfusion disease, hypoxia and other neurodegenerative diseases that are associated with inflammations, such as multiple sclerosis, amyotrophic lateral sclerosis and comparable sclerotic diseases, Parkinson's disease, Huntington's chorea, Korksakoff's syndrome, epilepsy, vomiting, sleep disturbances, schizophrenia, depression, migraine, hypoglycemia, dementia, such as, e.g., Alzheimer's disease, HIV dementia and presenile dementia.

They are also suitable for treating diseases of the cardiovascular system and for treating autoimmune and/or inflammatory diseases such as hypotension, ARDS (adult respiratory distress syndrome), sepsis or septic shock, rheumatoid arthritis, osteoarthritis, insulin-dependent diabetes mellitus (IDDM), inflammatory disease of the pelvis/intestine (bowel disease), meningitis, glomerulonephritis, acute and chronic liver diseases, diseases by rejection (for example allogenic heart, kidney or liver transplants) or inflammatory skin diseases such as psoriasis, etc. Based on their profile of action, the compounds according to the invention are very well suited for rearranging the peroxynitrite in harmless products.

The subject matter of this invention is also the use of compounds of general formula (D, characterized in that M stands for an Fe³⁺, Mn³⁺, Cu²⁺, Co³⁺, VO²⁺, Cr³⁺ or Ni²⁺-ion, and that are especially effective.

In addition, the subject matter of this invention is the use of porphyrin complex compounds of general formula I, characterized in that R² and R³ in each case stand for a —CONHNHK, —CONH(CH₂)₂NHK, —CONH(CH₂)₃NHK, —CONH(CH₂)₄NHK, or —CONH(CH₂)₂O(CH₂)₂NHK group.

The subject matter of this invention is also the use of porphyrin complex compounds of general formula (1), characterized in that R² and R³ in each case stand for a —CONHNHK.

These compounds are quite especially effective if K is a complexing agent of general formula (IIa):

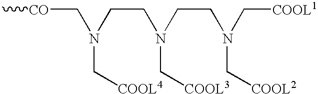

Additional subjects of this invention are in particular porphyrin complex compounds according to Formula (I), namely

{mu-[{16,16′-[Chloromanganese(III)-7,12-diethyl-3,8,13,17-tetramethylporphyrin-2,18-diyl]-bis[3,6,9-tris(carboxymethyl)-11,14-dioxo-3,6,9,12,13-pentaazahexadecanoato]}(8-)]}digadolinato(2-), -disodium,

{mu[{16,16′-[Chloroiron(III)-7,12-diethyl-3,8,13,17-tetramethylporphyrin-2,18-diyl]-bis[3,6,9-tris(carboxymethyl)-11,14-dioxo-3, 6, 9, 12, 13-pentaazahexa-decanoato]}(8-)]}-digadolinato(2-), -disodium,

{mu[{16,16′-[copper(II)-7,12-diethyl-3,8,13,17-tetramethylporphyrin-2,18-diyl]-bis[3,6,9-tris(carboxymethyl)-11,14-dioxo-3, 6, 9, 12,13-pentaazahexadecanoato]}(8-)]}-digadolinato(2-), -disodium.

The good water solubility of the agents according to the invention allows the production of highly-concentrated solutions, so as to keep the volume burden of the circulatory system within reasonable limits and to compensate for the dilution by bodily fluids. In addition, the agents according to the invention show not only a high stability in vitro, but also a surprisingly high stability in vivo, so that a release or an exchange of the ions, which are inherently toxic and not covalently bonded in the complexes, can be disregarded within the time that it takes for the contrast media to be completely excreted.

Surprisingly enough, the complexes according to the invention show a significantly higher relaxivity compared to the previously known, structurally similar compounds. Since the relaxivity can be regarded as a yardstick for the contrast medium action of a compound, a comparable, positive signal effect is possible even at a low dose with use of the complexes according to the invention in the area of NMR diagnosis. This significantly increases the safety margin, for which the product of relaxivity and compatibility can be considered as a guide value.

In addition, this invention relates to the use of the porphyrin complexes of general formula I according to claim 1 of the invention for diagnosis of diseases that comprise the group of the following diseases: ischemic reperfusion diseases, such as, e.g., stroke, head trauma and myocardial ischemia, sepsis, chronic or acute inflammation (such as, e.g., arthritis or inflammatory intestinal disease), adult respiratory stress syndrome, cancer, bronchio-pulmonary dysplasia, cardiovascular diseases, diabetes, multiple sclerosis, Parkinson's disease, familial amyotrophic lateral sclerosis and colitis and special neuronal diseases.

### DESCRIPTION OF THE FIGURES

FIG. 1 shows a ¹⁴NMR spectrum.

FIG. 2 shows the time-dependent degradation of peroxynitrite with and without a porphyrin catalyst, which was administered in a 100× deficit.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, all temperatures are set forth uncorrected in degrees Celsius and, all parts and percentages are by weight, unless otherwise indicated.

### EXAMPLES

1. Study of the Decomposition of Peroxynitrite with NMR-Spectroscopic Methods

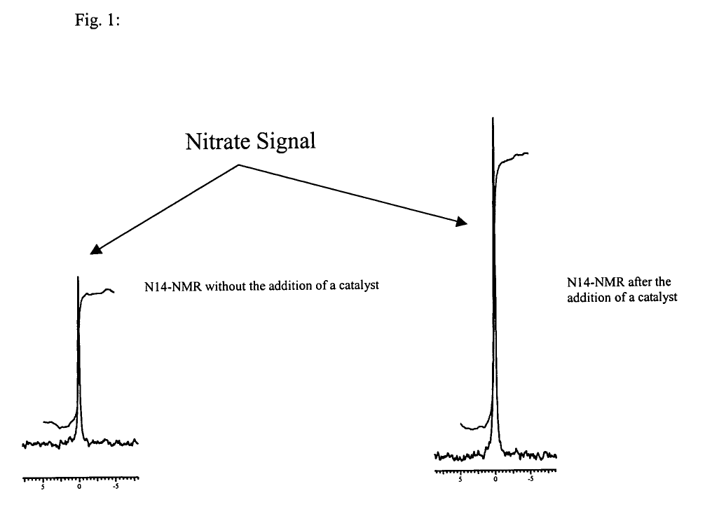

For the study, 3 samples are prepared, whereby one of the samples contains the original aqueous peroxynitrite solution without additives, and the others contain this solution in the same amount but with defined additions of a reference substance or the substance to be studied. From each sample, a 14-N spectrum with the same acquisition and processing parameters is recorded. The differences of the integrals of the nitrate signal between the treated and the untreated peroxynitrite solution indicates the increase in the nitrate owing to rearrangement of the peroxynitrite. Comparisons of the data between the substance to be studied and the reference compound allows a quantification of this process (FIG. 1).

2. Measurement of the Kinetics of the Conversion of Peroxynitrite to Nitrate by Means of UV-Spectrometry

Used are:

A UV-spectrometer

A stopped-flow device with a flanged cuvette

Laboratory device for volumetric works

Reagents (buffers, peroxynitrite solution, catalysts)

The content of peroxynitrite is determined from the concentrated peroxynitrite solution. A molar extinction coefficient of ε=1670 is taken as the baseline. The solution is diluted with water such that an absorption of about 1.6 at the observation wavelength of 301 nm is reached. The pH of the thus produced storage solution is not to fall below 11.

Corresponding to the set concentration of the peroxynitrite, a solution of the catalyst to be studied in the phosphate buffer is produced so that taking into consideration the given ratios of the stopped-flow device, the desired amounts of catalyst solution and peroxynitrite can be merged to bring about the reaction. The buffer of the catalyst solution must have sufficient capacity to be able to set and to hold the still strong alkaline peroxynitrite solution at the desired pH. The catalysts are added in a 100× deficit.

The metering sprayers of the stopped-flow unit are filled with the two solutions, and the cuvette that is located in the UV-spectrometer is filled therefrom. The absorption values are simultaneously measured at 301 nm. Because of the rearrangement of the peroxynitrite in the nitrate, absorption is decreased, assuming a stable value after some length of time. At this time, the rearrangement is terminated, and the data registration is brought to a halt.

The measurement data are analyzed, and the peroxynitrite concentration can be calculated from the resulting kinetic characteristics of the curve. These data are used to characterize the catalyst that is to be studied. To take into consideration the self-decomposition of the peroxynitrite, first the decomposition behavior of the peroxynitrite solution in any preparation of peroxynitrite that is used is determined in adding the buffer without catalyst content and whose characteristics are set in relation to those that are obtained from a measurement with catalyst.

Relative speed constant=Spontaneous decomposition of peroxynitrite at the described pH catalysed rearrangement

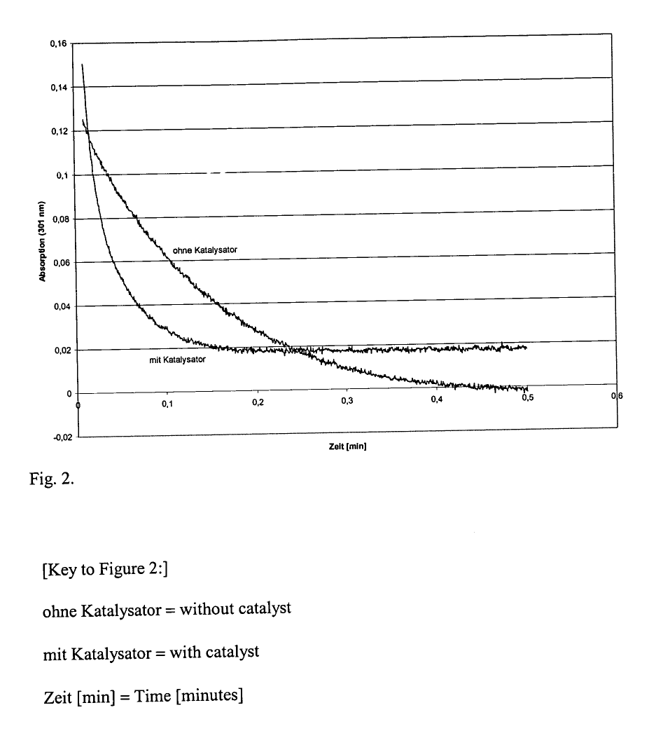

(FIG. 2).

3. SIN-1 Damage Assay with Neuronal Primary Cultures from the Cerebellum of Neonatal Rats

For in-vitro testing of substances for neuroprotection with respect to injuries that are induced by peroxynitrite, a primary culture of cells from the cerebellum of neonatal rats is applied. The measurement of cell death or survival of neurons in this culture is carried out indirectly by measurement of the reaction of the dye Alamar blue in its reduced fluorescent form. For injury, the peroxynitrite donor SIN-1 (3-morpholino-sydnonimine) is used.

To obtain the cells, Wistar rats (P8) are killed by decapitation, the cerebella are obtained, the meninges from the cerebellum are removed (HBSS (GIBCO, 14025-050) 4° C.), crushed and transferred into a 15 ml Falcon tube, the supernatant is suctioned off, and then the cerebella are trypsinized by means of adding 500 μl of trypsin-EDTA solution (GIBCO #2530-054)/cerebellum. After an incubation (20 minutes, 37° C.), the trypsinized cerebella are washed 3× with 10 ml of HBSS. A trituration by adding 500 μl of 0.05% DNAse1 (BOEHRINGER MANNHEIM, #14953000) per cerebellum follows. By means of a 5 ml pipette, then with a fire-polished Pasteur pipette and finally (if necessary) with an elongated, fire-polished Pasteur pipette, the cells are isolated and mixed with 10 ml of complete medium (100 ml of neurobasal (GIBCO #21103-049), 1 ml of B27 supplement (GIBCO #17504-044), 0.4 ml of Pen/Strep (10,000 IU/ml/10000 UG/ml) (GIBCO #15140-106), 0.8 ml of KCl-stock solution (MERCK, 1.04936.0500), and 1 ml of L-glutamine (100×=200 mmol) (GIBCO #15140-106). The isolated cells are then centrifuged off (10 minutes at 600 rpm), washed 1× with complete medium and resuspended with 20 ml of complete medium, counted and diluted to 2×10⁶/ml. Per hole of a 96-hole microtiter plate, 100 μl of complete medium is introduced and mixed with 100 μl of cell suspension (=div1=day 1 in vitro). The microtiter plates are coated beforehand as follows: 50 μl of poly-L-lysine (MW 70-105 kD) (SIGMA #P-6282) is applied per hole, and the plates are then incubated in an incubator for about 90 minutes. Before the cells are flattened out, the solution is suctioned off again and washed 2× with HBSS or with sterile bidistilled water. 24 hours after flattening out, the cells are damaged by adding SIN-1. Test substances are applied 1 hour before SIN-1 is added (individual concentration of 10 or 30 μM, or as a concentration series, CALBIOCHEM 567028). The measurement of the cell function is carried out on div2 (day2 in vitro) with Alamar blue (10 μl/well) (BIOSOURCE INT., DAL 1100). After a 3-hour incubation, the measurement in the fluorescence reader follows (Victor, Wallac Company, Extinction 544 nm/emission 590 nm). IC50 values are calculated with the Excel Plug-in Xlfit.

The results from Examples 2 and 3 are indicated in the following table.

| | | | Cell Toxicity Assay |
|---|---|---|---|
| Example | Complex | Speed Constant | ED50 [μM] |
| 1 | 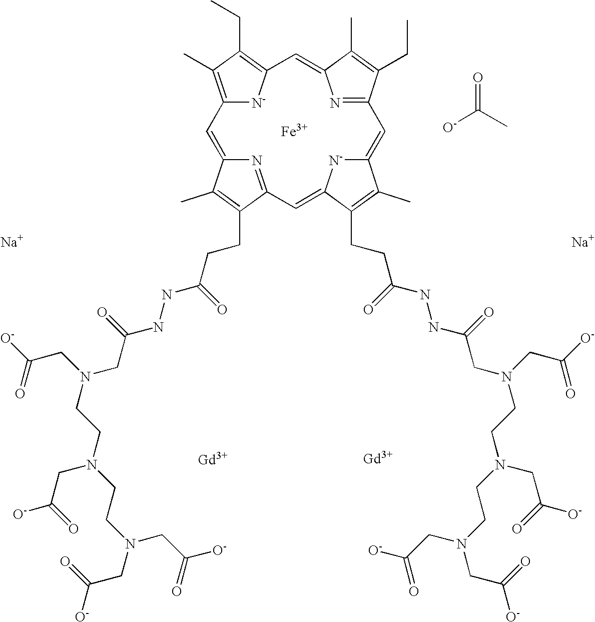 | 3.75 | >30 |
| 2 | 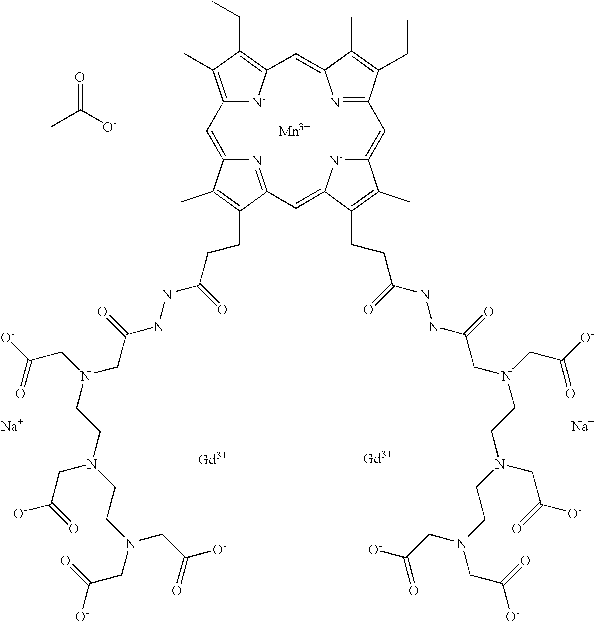 | 1.61 | 9.6 |

The entire disclosures of all applications, patents and publications, cited herein and of corresponding German Patent Application No. 102 40 343.0-44, filed Aug. 27, 2002, and U.S. Provisional Application Serial No. 60/406,985, filed Aug. 30, 2002 and U.S. Provisional Application Serial No. 60/461,417 filed on Apr. 10, 2003 are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Use of a porphyrin complex that consists of a ligand of general formula I 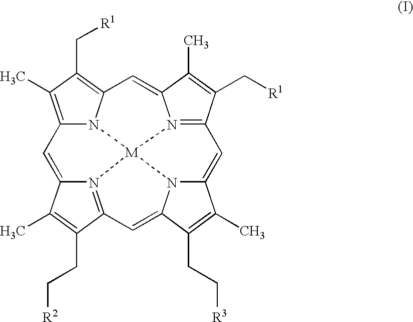
as well as at least one ion of an element of atomic numbers 20-32, 37-39, 42-51 or 57-83, in which
M stands for a paramagnetic ion,
R¹ stands for a hydrogen atom, for a straight-chain C₁-C₆-alkyl radical, a C₇-C₂-aralkyl radical or for a group OR′, in which
R′ is a hydrogen atom or a C₁-C₃-alkyl radical,
R² stands for R³ a group —CO-Z or a group —(NH)ₒ—(A)_{q}—NH-D, in which
Z is a group —OL, with L meaning an inorganic or organic cation or a C₁-C₄-alkyl radical,
A means a phenylenoxy group or a C₁-C₁₂-alkylene group or a C₇-C₁₂ aralkylene group that is interrupted by one or more oxygen atoms, o and q, independently of one another, mean the number 0 or 1, and
D means a hydrogen atom or a group —CO-A-(COOL)ₒ-(H)ₘ, with m equal to 0 or 1, provided that the sum of m and o is equal to 1,
R³ stands for a group —(C=Q)(NR⁴)ₒ-(A)_{q}—(NR⁵)—K,
in which Q stands for an oxygen atom or for two hydrogen atoms,
R⁴ means a group -(A)_{q}-H, and
K means a complexing agent of general formula (IIa), (IIIb), (IIc), (IId) or (IIe), whereby R⁵, for the case that K is a complexing agent of formula (IIa), has the same meaning as R⁴, and R⁵, for the case that K is a complexing agent of formula (IIb), (IIc), (IId) or (IIe), has the same meaning as D,
provided that a direct oxygen-nitrogen bond is not allowed,
and K stands for a complexing agent of general formula (IIa), (IIb), (IIc), (IId), (IIe) or (IIf) 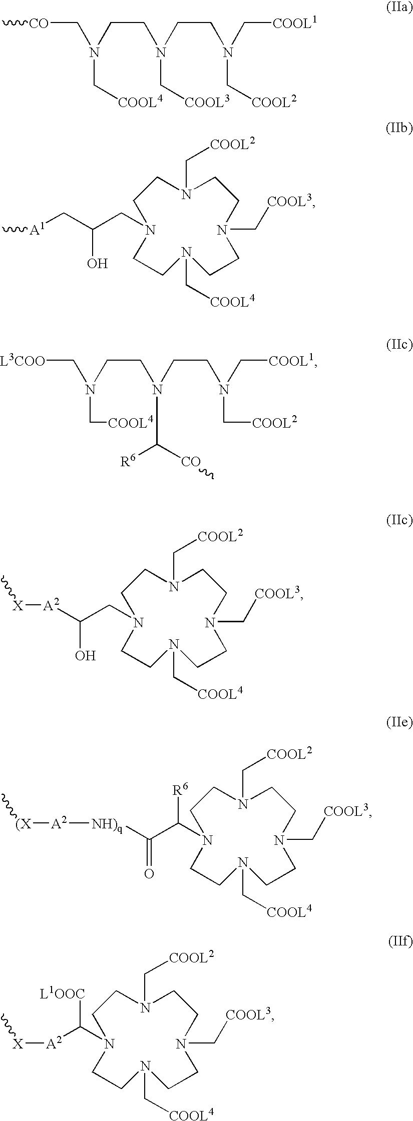
in which
q has the above-indicated meaning,
A¹ has the meaning that is indicated for A,
R⁶ stands for a hydrogen atom, a straight-chain or branched C₁-C₇-alkyl group, a phenyl group or a benzyl group,
A² stands for a phenylene-, —CH₂—NHCO—CH₂—CH(CH₂COOH)—C₆H₄—β-, —C₆H₄—O—(CH₂)₀₋₅-β, or —C₆H₄—(OCH₂CH₂)₀₋₁-N(CH₂COOH)—CH₂-β group, or a C₁-C₁₋₂-alkylene group or a C₇-C₁₂-alkylene group that is optionally interrupted by one or more oxygen atoms, 1 to 3-NHCO groups, or 1 to 3-CONH groups, and/or substituted with 1 to 3 (CH₂)₀₋₅COOH groups, whereby β stands for the binding site to X,
X stands for a —CO— or NHCS-group, and
L¹, L², L³ and L⁴, independently of one another, stand for a hydrogen atom or a metal ion equivalent of an element of the above-mentioned atomic number, provided that at least two of these substituents stand for metal ion equivalents and that other anions are present to compensate for optionally present charges in the metalloporphyrin, and in which free carboxylic acid groups that are not required for complexing can also be present as salts with physiologically compatible inorganic and/or organic cations or as esters or as amides, for the production of a pharmaceutical agent for treatment and prophylaxis of radical-mediated cell injuries.

2. Use of the porphyrin complexes according to claim 1 for the production of a pharmaceutical agent for the treatment and prophylaxis of diseases that are caused by peroxynitrite-mediated reactions and that are weakened and/or treated by the increase in the conversion rate of peroxynitrite.

3. Use of the porphyrin complexes according to claim 1 or 2 for the treatment and prophylaxis of diseases that comprise the group of the following diseases:
ischemic reperfusion diseases, such as, e.g., stroke, head trauma and myocardial ischemia, sepsis, chronic or acute inflammation (such as, e.g., arthritis or inflammatory intestinal disease), adult respiratory stress syndrome, cancer, bronchio-pulmonary dysplasia, cardiovascular diseases, diabetes, multiple sclerosis, Parkinson's disease, familial amyotrophic lateral sclerosis and colitis and special neuronal diseases.

4. Use of the porphyrin complexes of general formula I according to claim 1, characterized in that M stands for an Fe³⁺, Mn³⁺, Cu²⁺, Co³⁺, VO²⁻, Cr³⁺ or Ni²⁺ ion.

5. Use of the porphyrin complex compounds of general formula I according to claim 1 or 4, wherein R² and R³ in each case stand for a —CONHNHK, —CONH(CH₂)₂NHK, —CONH(CH₂)₃NHK, —CONH(CH₂)₄NHK, or —CONH(CH₂)₂O(CH₂)₂NHK group.

6. Use of the porphyrin complexes according to claim 1, 4 or 5, wherein R² and R³ in each case stand for a —CONHNHK.

7. Compounds according to claim 6, wherein K is a complexing agent of general formula (IIa)

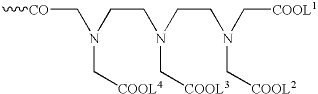

8. Use of the porphyrin complex compounds according to formula 1 of claim 1, namely
{mu-[{16,16′-[Chloromanganese(III)-7,12-diethyl-3,8,13,17-tetramethylporphyrin-2,18-diyl]-bis[3,6,9-tris(carboxymethyl)-11,14-dioxo-3,6,9,12,13-pentaazahexadecanoato]}(8-)]}digadolinato(2-), -disodium,
{mu[{16,16′-[chloroiron(III)-7,12-diethyl-3,8,13,17-tetramethylporphyrin-2,18-diyl]-bis[3,6,9-tris(carboxymethyl)-11,14-dioxo-3,6,9,12,13-pentaazahexadecanoato])}-(8-)]}-digadolinato(2-),-disodium,
{mu[{16,16′-[copper(II)-7,12-diethyl-3,8,13,17-tetramethylporphyrin-2,18-diyl]-bis[3,6,9-tris(carboxymethyl)-11,14-dioxo-3,6,9,12,13-pentaazahexadecanoato]}(8-)]}-digadolinato(2-),-disodium

9. Use of the porphyrin complexes of general formula I according to claim 1 for the diagnosis of diseases that comprise the following groups:
a. Ischemic reperfusion diseases
b. Acute and chronic inflammatory diseases
c. Autoimmune diseases
d. Neurodegenerative and neuroregenerative diseases.
